(19) United States
(12) Patent Application Publication
(10) Pub. No.: US 2023/0382847 A1
(43) Pub. Date: Nov. 30, 2023

(54) METHOD OF FORMING A CHELATING COMPOSITION

(71) Applicant: NOURYON CHEMICALS INTERNATIONAL B.V., Amsterdam (NL)

(72) Inventors: Edwin Rudolf Antony BANG, Arnhem (NL); Martin HEUS, Arnhem (NL); Tjerk Oedse BOONSTRA, Duiven (NL)

(73) Assignee: NOURYON CHEMICALS INTERNATIONAL B.V., Amsterdam (NL)

(21) Appl. No.: 18/324,285

(22) Filed: May 26, 2023

Related U.S. Application Data
(60) Provisional application No. 63/365,491, filed on May 30, 2022.
(60) Provisional application No. 63/365,492, filed on May 30, 2022.
(60) Provisional application No. 63/365,493, filed on May 30, 2022.

(51) Int. Cl. C07C 227/16

(52) U.S. Cl. CPC C07C 227/16

(57) ABSTRACT

A method of forming a chelating composition includes the steps of: providing a first mixture comprising water and a tetrasodium salt of glutamic acid N,N-diacetic acid and having a pH of greater than about 10; and acidifying the first mixture via ion exchange and/or bipolar membrane electrodialysis to reduce a pH of the chelating composition to a pH of at least about 9; wherein the chelating composition comprises the tetrasodium salt of glutamic acid N,N-diacetic acid in an amount of at least about 50 weight percent based on a total weight of the chelating composition as determined using an Fe-Total Sequestering Value; and wherein the chelating composition has a viscosity of less than about 1350 mPa·s measured at about 5° C., or less than about 350 mPa·s measured at about 20° C., each using a Brookfield DV II plus viscometer with spindle S18 and a temperature controlled bath.

### CROSS-REFERENCE TO RELATED APPLICATIONS

This application claims the benefit of U.S. Provisional Application No. 63/365,491, filed May 30, 2022, U.S. Provisional Application No. 63/365,492, filed May 30, 2022, and U.S. Provisional Application No. 63/365,493, filed May 30, 2022, each of which is hereby expressly incorporated herein in its entirety by reference.

### TECHNICAL FIELD

The present disclosure generally relates to a method of forming a chelating composition comprising the tetrasodium salt of glutamic acid N,N-diacetic acid (GLDA). The chelating composition has a low viscosity while including a surprisingly high weight percent of the GLDA.

### BACKGROUND

Several sodium salts of the chelating compound glutamic acid N,N-diacetic acid (GLDA) are known in the art. For instance, U.S. Pat. No. 8,455,682 describes the synthesis of GLDA tetrasodium salt using HCN and formaldehyde and glutamic acid or monosodium glutamate. The method makes use of ambient or elevated temperature (10-100° C.; especially 20-80° C.) and dosing times of 10 minutes to 10 hours but especially 30 minutes to 3 hrs. The process described is a two-step process in which first one equivalent formaldehyde is dosed to the pre-charged glutamate and in a 2nd step HCN and the other equivalent of formaldehyde are dosed.

Another HCN based GLDA process is described in WO 2009/024518, wherein examples result in mixed sodium/potassium salts of GLDA; GLDA-NaₓK_{y} (x+y=4). In this reference, a two-step process at ambient or slightly elevated temperature is used. The first step describes the synthesis of a Schiff-base before applying a second dosing step in which remaining amounts of formaldehyde/HCN are added.

Typically, in the art, a yield on glutamate in GLDA-Na₄ processes using HCN/formaldehyde technology is from about 89 to about 94%. However, the art does not describe why the yield is limited. The only by-product which is typically described is NTA-Na₃ (nitrilotriacetic acid sodium salt) which is present at a level below about 0.1 wt % which does not explain why the yield based on glutamate/glutamic acid conversion is about 90%

It is known in the art that handling GLDA tetrasodium salt solutions in high concentrations has some major drawbacks when used on a commercial scale. An important problem is the high viscosity of concentrated solutions of GLDA tetrasodium salt which makes such a solution difficult to handle. The high viscosity limits the practical concentration to maximum values of about 47 wt %. Today, at concentrations above about 55 to about 60 wt %, the GLDA tetrasodium salt tends to become a paste, making it necessary to heat containers before the product can be poured out. GLDA-Na₄ solutions do not typically crystallize when concentrated. It is simply the increasing viscosity that prevents use of higher concentrations.

Moreover, aqueous GLDA-Na₄ solutions tend to be corrosive to aluminum mainly due to a free caustic by-product in the GLDA solution. This therefore limits applications to corrosive-proof installations. These problems become particularly relevant when using GLDA-Na₄ solutions in large-scale productions having aluminum parts such as spray driers, especially when large containers of about 1,000 KG or more are used. Accordingly, there remains an opportunity for improvement.

### BRIEF SUMMARY

This disclosure provides a method of forming a chelating composition, the method comprising the steps of: providing a first mixture comprising water and the tetrasodium salt of glutamic acid N,N-diacetic acid and having a pH of greater than about 10; and acidifying the first mixture via ion exchange and/or bipolar membrane electrodialysis to reduce a pH of the chelating composition to a pH of at least about 9; wherein the chelating composition comprises the tetrasodium salt of glutamic acid N,N-diacetic acid in an amount of at least about 45 weight percent based on a total weight of the chelating composition as determined using an Fe-Total Sequestering Value; and wherein the chelating composition has a viscosity of less than about 1350 mPa·s measured at about 5° C., or less than about 350 mPa·s measured at about 20° C., each using a Brookfield DV II plus viscometer with spindle S18 and a temperature controlled bath.

In various embodiments, this disclosure increases sustainability and the ability to transport and package of aqueous chelant solutions using solutions that still allows handling at room temperature without being too viscous, as would be appreciated by one of skill in the art.

### BRIEF DESCRIPTION OF THE DRAWINGS

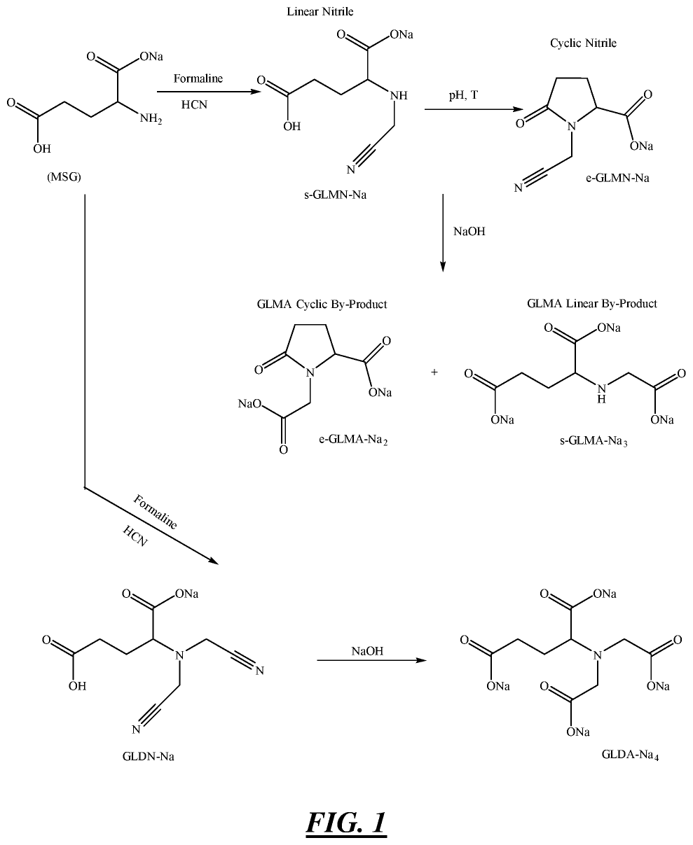

The present disclosure will hereinafter be described in conjunction with the following FIGURE, wherein FIG. 1 is a synthetic scheme showing formation of the tetrasodium salt of glutamic acid N,N-diacetic acid and various by-products.

### DETAILED DESCRIPTION

The following detailed description is merely exemplary in nature and is not intended to limit the current chelating composition and/or method of forming. Furthermore, there is no intention to be bound by any theory presented in the preceding background or the following detailed description.

Embodiments of the present disclosure are generally directed to chelating compositions and methods for forming the same. For the sake of brevity, conventional techniques related to chelating compositions may not be described in detail herein. Moreover, the various tasks and process steps described herein may be incorporated into a more comprehensive procedure or process having additional steps or functionality not described in detail herein. In particular, various steps in the manufacture of chelating compositions are well-known and so, in the interest of brevity, many conventional steps will only be mentioned briefly herein or will be omitted entirely without providing the well-known process details. In this disclosure, the terminology “about” can describe values ±0.1, 0.5, 1, 2, 3, 4, 5, 6, 7, 8, 9, or 10%, in various embodiments. Moreover, it is contemplated that, in various non-limiting embodiments, all values set forth herein may be alternatively described as approximate or “about.” It is contemplated that all isomers and chiral options for each compound described herein are hereby expressly contemplated for use herein.

Moreover, it is contemplated that, in various non-limiting embodiments, it is to be appreciated that all numerical values as provided herein, save for the actual examples, are approximate values with endpoints or particular values intended to be read as “about” or “approximately” the value as recited.

Throughout this disclosure, the terminology percent “actives” is well recognized in the art and means the percent amount of active or actual compound or molecule present as compared to, for example, a total weight of a diluted solution of a solvent and such a compound. Some compounds, such as a solvent, are not described relative to a percent actives because it is well known to be approximately 100% actives. Any one or more of the values describe herein may be alternatively described as percent actives as would be understood by the skilled person.

In various embodiments, the terminology “free of” describes embodiments that include less than about 5, 4, 3, 2, 1, 0.5, or 0.1, weight percent (or weight percent actives) of the compound or element at issue using an appropriate weight basis as would be understood by one of skill in the art. In other embodiments, the terminology “free of” describes embodiments that have zero weight percent of the compound or element at issue.

The terminology “consists essentially of” may describe various non-limiting embodiments that are free of one or more optional compounds described herein and/or free of one or more polymers, surfactants, additives, solvents, etc.

It is to be understood that the subscripts of polymers are typically described as average values because the synthesis of polymers typically produces a distribution of various individual molecules.

The polymers and compositions disclosed herein may suitably comprise, consist of, or consist essentially of the components, elements, and process delineations described herein. The embodiments illustratively disclosed herein suitably may be practiced in the absence of any element which is not specifically disclosed herein.

In various embodiments, glutamic acid, monosodium glutamate and/or disodium glutamate or mixtures thereof can be starting materials for producing a GLDA sodium salt. It is known by the skilled person that, for example, glutamic acid also needs caustic to form a monosodium salt. For example, glutamic acid alone is typically not present because it cannot form a sodium salt of a nitrile. However, a mixture of glutamic acid, MSG and disodium glutamic acid may be utilized. For example, a two-step reaction can be utilized wherein glutamate or glutamic acid are converted into GLDA. A first reaction may be the synthesis of a nitrile while a second reaction may be hydrolysis/saponification of a resulting nitrile.

In various embodiments, this disclosure provides a highly pure aqueous solution of a sodium salt of the chelating compound GLDA (glutamic acid N,N-diacetic acid) and a process for making such solution e.g. using a waste-free process without any required purification step.

In other embodiments, this disclosure provides an alternative for the aqueous solutions of the tetrasodium salt of commercially available GLDA that have an approximately equal viscosity to currently available commercial products but higher concentrations of GLDA than those same commercial products having no or less corrosive properties. In other embodiments, the disclosure provides a product that can be handled in containers at ambient temperature wherein a solution has high purity and can be made using a waste free or waste-minimized process.

In still other embodiments, the disclosure provides aqueous solutions of sodium salts of GLDA of varying concentrations. In some embodiments, there is no need to use mixed sodium/potassium salts of GLDA which is a benefit as KOH is not as readily available and more expensive. Moreover, this can also reduce viscosity.

Typically, wherever pH is described herein, it is the pH of a 1 wt % actives solution.

### Chelating Composition

In various embodiments, this disclosure provides a chelating composition having a pH of at least about 9 or 9.5 measured as an about 1 wt % solution. In various embodiments, the pH is from about 8 to about 14, about 8.5 to about 14, about 9 to about 14, about 9.5 to about 14, about 9.5 to about 12.5, about 9.5 to about 12, about 9.5 to about 11.5, about 9.5 to about 11, about 9.5 to about 10.5, about 9.5 to about 10, about 10 to about 12, about 10 to about 11.5, about 10 to about 11, about 11 to about 12, about 11 to about 11.5, or about 11.5 to about 12, measured as an about 1 wt % solution. Typically, pH is important because the skilled person appreciates that a pH value that is too high will be corrosive towards aluminum. Furthermore, a pH that is too high indicates the presence of free caustic or NaOH even in small concentrations (wt %) which will increase viscosity of the composition because its molecular weight is small relative to the large molecular weight of the tetrasodium salt of glutamic acid N,N-diacetic acid. In various non-limiting embodiments, all values and ranges of values, both whole and fractional, including and between the aforementioned values, are hereby expressly contemplated for use herein.

The chelating composition may be, include, consist essentially of, or consist of, water and a tetrasodium salt of glutamic acid N,N-diacetic acid (GLDA). In one embodiment, the terminology “consist essentially of” describes embodiments that are free of, or include less than 5, 4, 3, 2, 1, 0.5, or 0.1, weight percent based on a total weight of the composition, of one or more byproducts known to be produced when forming GLDA, such as those described below. In various non-limiting embodiments, all values and ranges of values, both whole and fractional, including and between the aforementioned values, are hereby expressly contemplated for use herein.

The synthesis of GLDA (and GLDN, which is a nitrile precursor of GLDA) is not 100% selective. The reaction of glutamic acid or monosodium glutamate or mixtures thereof with formaldehyde and cyanide results after hydrolysis of the nitrile solution in a small amount of glycolate and formate formation but, as was recently discovered, also glutamic acid monoacetic acid and a byproduct called 1-pyrrolidineacetic acid, 2-carboxy-5-oxo sodium salt which can be described as GLMA cyclic. The GLMA cyclic does not sequester metal ions and contributes to viscosity, cost, yield loss and density. Another byproduct, GLMA linear, is a very weak chelating agent and also contributes to viscosity, density and yield loss. After identifying the by-products GLMA linear and GLMA cyclic, it became clear why historical reported yields on glutamate conversion to GLDA have been reported at a maximum of 94% (more commonly a 90% yield is reported). A high conversion of MSG or the conversion of a mixture of MGS-glutamic acid to GLDA is possible by utilizing additional HCN and formaldehyde. However, one possible consequence is that the NTA level may be too high (>0.1 wt %) due to formaldehyde and HCN in a saponification step reacting with NH₃ that is formed by the hydrolysis of the nitrile functionality. A challenge solved herein is to achieve a high conversion/high yield while keeping NTA at or below about 0.1 wt %

GLMA, which is glutamic acid mono acetic acid, is typically the main byproduct in the production of GLDA. Two GLMA isomers can be formed by saponification of corresponding nitrile precursors, which can be described as GLMN linear/open and GLMN cyclic. Structures of GLMN linear and GLMN cyclic are set forth in FIG. 1.

The synthesis of GLDN, as a nitrile precursor for GLDA, in high yield is a challenge. Since the nitrile mono-adduct GLMN can form a ring structure which will not open any more once it is formed, the conditions during GLDN nitrile synthesis should be controlled such that GLMN concentration will be low and only present for a short time. Typically, the higher the GLMN linear concentration and the longer the residence time of reaction, the more cyclic GLMN will be formed. Temperature and pH are typically important as well. At higher temperatures, reactions occur faster. This is not only true for the synthesis of GLDN but also for by-products such as cyclic GLMN. The most favorable reaction conditions are those that result in GLDN formation but limit cyclic GLMN formation.

GLDA is unique among chelating agents because its nitrile precursor, used to synthesize high purity chelating agent, can form a ring structure. Such structures normally include 5 or 6-atoms. When a mono-nitrile is formed, the hydrogen atom bonded to the nitrogen atom can still react with the carboxylic acid group from the splitting of water resulting in the ring structure referred to as cyclic GLMN-Na. The structure of the mono-nitrile allows the formation of a ring structure which once formed will never become a GLDA sodium salt. A well-known product like MGDA, which is also a chelating agent, doesn't face this particular challenge because the mono-nitrile precursor of MGDA (methylglycine monoacetonitrile) is incapable of forming a ring structure.

In various embodiments a low reaction temperature in the nitrile synthesis will reduce the formation of cyclic GLMN. A short residence time of GLMN linear is important too as it reduces the possibility of cyclic GLMN formation. One problem to overcome is the strong exothermic behavior of the nitrile synthesis. At the moment HCN is dosed, significant heat is generated thereby increasing the reaction temperature. Efficient cooling is required. The heat developed depends on the amount of HCN but in case HCN is precharged before formaldehyde on the amount of formaldehyde that is dosed. Slower dosing helps to control the reaction temperature by cooling but it increases the residence time of high GLMN concentrations present during the GLDN synthesis. As a result, more cyclic GLMN is produced.

Referring back, in various embodiments, the chelating composition includes water present in an amount of from about 35 to about 50, about 35 to about 45, about 35 to about 40, about 40 to about 50, about 40 to about 45, about 41 to about 45, about 41 to about 44, about 41 to about 43, about 41 to about 42, or about 41, 42, 43, 44, or 45, weight percent when the GLDA is about 55 wt % concentrated having slightly more or less byproducts. As would be understood by those of skill in the art, the aforementioned values could change if the weight percent of the GLDA is changed. All of these possibilities are hereby expressly contemplated herein in various non-limiting embodiments. In other non-limiting embodiments, all values and ranges of values, both whole and fractional, including and between the aforementioned values, are hereby expressly contemplated for use herein.

The tetrasodium salt of the glutamic acid N,N-diacetic acid (GLDA) is present in an amount of at least about 45 weight percent based on a total weight of the chelating composition as determined using an Fe-Total Sequestering Value. In various embodiments, this weight percent is from about 45 to about 70, about 50 to about 70, about 55 to about 70, about 60 to about 65, about 50 to about 60, about 50 to about 65, about 50 to about 55, or about 55 to about 60, as determined using an Fe-Total Sequestering Value. In other embodiments, this weight percent is about 52 to about 60, about 53 to about 59, about 54 to about 58, about 55 to about 57, or about 55 to about 56, as determined using an Fe-Total Sequestering Value. In various non-limiting embodiments, all values and ranges of values, both whole and fractional, including and between the aforementioned values, are hereby expressly contemplated for use herein.

The method of determining weight percent based on the Fe-Total Sequestering Value is known in the art, e.g. as described by Dow Chemical relative to use of Versene™ chelating agents and as set forth in Appendix A.

Referring back, the chelating composition has a viscosity of less than about 1350 mPa·s measured at about 5° C. or less than about 350 mPa·s at about 20° C., each as measured using a Brookfield DV II plus viscometer with spindle S18 and a temperature controlled bath. In various embodiments, the viscosity is measured at a temperature of about 5° C. using a Brookfield DV II plus viscometer with spindle S18 and a temperature controlled bath. In such embodiments, the viscosity may be less than about 1350, 1300, 1250, 1200, 1150, 1100, 1050, 1000, mPa·s etc. In other embodiments, the viscosity is measured at a temperature of about 20° C. using a Brookfield DV II plus viscometer with spindle S18 and a temperature controlled bath. In such embodiments, the viscosity may be less than about 350, 300, 250, 200, 150, 100, mPa·s etc. In various non-limiting embodiments, all values and ranges of values, both whole and fractional, including and between the aforementioned values, are hereby expressly contemplated for use herein.

In other embodiments, the chelating composition can be described as non-corrosive as determined by United Nations Standard Recommendations on the transport of the dangerous goods, Manual of tests and criteria; ST/SG/AC.10/11/Rev 4. As is known in the art, this standard involves tested steel types of P3 (ISO 2604 (IV)-1975 or a comparable type, for aluminum 7075-T6 or AZ5GU-T6 is required. Reliable measurement require a volume/surface area of >0.50 ml/mm². Materials used in this investigation are carbon steel SAE 1020 and aluminum 7075 T6. In various non-limiting embodiments, all values and ranges of values, both whole and fractional, including and between the aforementioned values, are hereby expressly contemplated for use herein.

In one embodiment, the viscosity is less than about 1200 mPa·s measured at about 5° C. using a Brookfield DV II plus viscometer with spindle S18 and a temperature controlled bath and the composition is classified as non-corrosive as determined by United Nations Standard Recommendations on the transport of the dangerous goods, Manual of tests and criteria; ST/SG/AC.10/11/Rev 4. In various non-limiting embodiments, all values and ranges of values, both whole and fractional, including and between the aforementioned values, are hereby expressly contemplated for use herein.

In other embodiments, the chelating composition has a density greater than about 1300, 1350, 1400, 1450, or even 1500, kg/m³. In such embodiments, shipping volumes are highly attractive as shipping costs are reduced and CO₂ footprints are reduced. In various non-limiting embodiments, all values and ranges of values, both whole and fractional, including and between the aforementioned values, are hereby expressly contemplated for use herein.

In another embodiment, the pH of the chelating composition is from about 10 to about 10.5 measured as an about 1 wt % solution. In another embodiment, the pH of the chelating composition is from about 8 to about 10.5 measured as an about 1 wt % solution. In another embodiment, the pH of the chelating composition is from about 8.5 to about 10.5 measured as an about 1 wt % solution. In another embodiment, the pH of the chelating composition is from about 9 to about 10.5 measured as an about 1 wt % solution. In another embodiment, the pH of the chelating composition is from about 9.5 to about 10.5 measured as an about 1 wt % solution. In various non-limiting embodiments, all values and ranges of values, both whole and fractional, including and between the aforementioned values, are hereby expressly contemplated for use herein.

The aforementioned chelating composition can be formed by any method known in the art and is not limited. However, in one embodiment, the chelating composition is formed from combining: a first mixture comprising water and the tetrasodium salt of glutamic acid N,N-diacetic acid having a pH of greater than about 10; and a second mixture comprising water and the sodium salt of glutamic acid N,N-diacetic acid having a pH of less than about 7. In other embodiments, this pH can be less than about 8.5, 7, 7.5, 7, 6.5, 6, 5.5, or 5. As would be understood by the skilled person, weak ion exchange resins delivering a slightly higher pH solution can also be used too wherein using a less acidic product typically requires more volume/product to reduce the pH to target value and that typically changes the ratio non-acidified GLDA:acidified GLDA. In various non-limiting embodiments, all values and ranges of values, both whole and fractional, including and between the aforementioned values, are hereby expressly contemplated for use herein.

The first mixture may be, include, consist essentially of, or consist of, the water and the tetrasodium salt of glutamic acid N,N-diacetic acid. In various embodiments, the terminology “consist essentially of” describes embodiments that are free of, or include less than 5, 4, 3, 2, 1, 0.5, or 0.1, weight percent based on a total weight of the composition, of one or more byproducts known to be produced when forming GLDA, such as those described herein. In various non-limiting embodiments, all values and ranges of values, both whole and fractional, including and between the aforementioned values, are hereby expressly contemplated for use herein.

In the first mixture, the amount of water is typically from about 0.1 to about 80, about 0.1 to about 75, about 0.1 to about 70, about 0.1 to about 65, about 0.1 to about 60, about 0.1 to about 55, about 0.1 to about 50, about 5 to about 45, about 10 to about 40, about 15 to about 35, about 20 to about 30, about 25 to about 30, about 0.1 to about 5, about 0.5 to about 5, or about 1, 2, 3, 4, or 5, weight percent based on a total weight percent of the first mixture. Also in the first mixture, the pH is typically greater than about 10, 10.5, 11, 11.5, 12, etc. measured as an about 1 wt % solution. In various embodiments, this pH is from about 11 to about 12 measured as an about 1 wt % solution. The pH of the first mixture is typically important for the same reasons as described above. Moreover, in a formaldehyde/cyanide based manufacturing method involving saponification, this type of method in the last step delivers a GLDA solution having a small amount of free caustic. The excess caustic is needed to ensure all nitrile groups will be fully hydrolyzed. However, after hydrolysis, the free caustic should remain limited as otherwise neutralization by acidic GLDA will require more of the acidified GLDA product. Furthermore, a high free caustic level makes the GLDA solution more viscous. In various non-limiting embodiments, all values and ranges of values, both whole and fractional, including and between the aforementioned values, are hereby expressly contemplated for use herein.

Also in the first mixture, the amount of the tetrasodium salt of glutamic acid N,N-diacetic acid is typically from about 30 to about 60, about 35 to about 55, about 40 to about 50, or about 45 to about 50, weight percent based on a total weight percent of the first mixture. In various non-limiting embodiments, all values and ranges of values, both whole and fractional, including and between the aforementioned values, are hereby expressly contemplated for use herein.

The second mixture may be, include, consist essentially of, or consist of, the water and the sodium salt of glutamic acid N,N-diacetic acid. In various embodiments, the terminology “consist essentially of” describes embodiments that are free of, or include less than 5, 4, 3, 2, 1, 0.5, or 0.1, weight percent based on a total weight of the composition, of one or more byproducts known to be produced when forming GLDA, such as those described below. In various non-limiting embodiments, all values and ranges of values, both whole and fractional, including and between the aforementioned values, are hereby expressly contemplated for use herein.

In the second mixture, the amount of water is typically from about 35 to about 60, about 40 to about 55, or about 45 to about 50, weight percent based on a total weight percent of the second mixture. Also in the second mixture, the pH is typically less than about 7, 6.5, 6, 5.5, 5, 4.5, 4, 3.5, 3, etc. measured as an about 1 wt % solution. In various embodiment, this pH is from about 3 to about 4, measured as an about 1 wt % solution. The pH of the second mixture is important as described above. Moreover, at too high of pH, the second mixture requires a high ratio of acidic GLDA to get pH ˜9-10 which would require a larger volume of GLDA to be acidified In various non-limiting embodiments, all values and ranges of values, both whole and fractional, including and between the aforementioned values, are hereby expressly contemplated for use herein.

In various embodiments, the second mixture is acidified via ion exchange and/or bipolar membrane electrodialysis. This process may be or include any one or more steps described in at least U.S. Pat. No. 8,551,312B2 which is expressly incorporated herein in its entirety in various non-limiting embodiments.

Also in the second mixture, the amount of the sodium salt of glutamic acid N,N-diacetic acid (typically expressed as the tetrasodium salt or partially acidified GLDA sodium salt) is typically from about 15 to about 50, about 20 to about 35, or about 25 to about 30, weight percent based on a total weight of the chelating composition as determined using an Fe-Total Sequestering Value. In various non-limiting embodiments, all values and ranges of values, both whole and fractional, including and between the aforementioned values, are hereby expressly contemplated for use herein.

### Method of Forming a Chelating Composition

This disclosure also provides a method of forming a chelating composition that includes the steps of providing a first mixture comprising water and the tetrasodium salt of glutamic acid N,N-diacetic acid and having a pH of greater than about 8, 8.5, 9, 9.5, or 10.

The first mixture may be, include, consist essentially of, or consist of, the water and the tetrasodium salt of glutamic acid N,N-diacetic acid. In various embodiments, the terminology “consist essentially of” describes embodiments that are free of, or include less than 5, 4, 3, 2, 1, 0.5, or 0.1, weight percent based on a total weight of the composition, of one or more byproducts known to be produced when forming GLDA, such as those described herein. In various non-limiting embodiments, all values and ranges of values, both whole and fractional, including and between the aforementioned values, are hereby expressly contemplated for use herein.

In the first mixture, the amount of water is typically from about 5 to about 80, about 5 to about 75, about 5 to about 70, about 5 to about 65, about 5 to about 60, about 5 to about 55, about 5 to about 50, about 5 to about 45, about 10 to about 40, about 15 to about 35, about 20 to about 30, about 25 to about 30, about 0.1 to about 5, about 0.5 to about 5, or about 1, 2, 3, 4, or 5, weight percent based on a total weight percent of the first mixture. Also in the first mixture, the pH is typically greater than about 8, 8.5, 9, 9.5, 10, 10.5, 11, 11.5, 12, etc. measured as an about 1 wt % solution. In various embodiments, this pH is from about 11 to about 12 measured as an about 1 wt % solution. The pH of the first mixture is typically important for the same reasons as described above. Moreover, in a formaldehyde/cyanide based manufacturing method involving saponification, this type of method in the last step delivers a GLDA solution having a small amount of free caustic which should remain limited as otherwise neutralization by acidic GLDA will require more of the acidified GLDA product. In various non-limiting embodiments, all values and ranges of values, both whole and fractional, including and between the aforementioned values, are hereby expressly contemplated for use herein.

Also in the first mixture, the amount of the tetrasodium salt of glutamic acid N,N-diacetic acid is typically from about 30 to about 60, about 35 to about 55, about 40 to about 50, or about 45 to about 50, weight percent based on a total weight percent of the first mixture. In various embodiments, the tetrasodium salt is present in the first mixture in an amount of from about 60 to about 70 weight percent based on a total weight of the chelating composition as determined using an Fe-Total Sequestering Value. In various non-limiting embodiments, all values and ranges of values, both whole and fractional, including and between the aforementioned values, are hereby expressly contemplated for use herein.

The method also includes the step of acidifying the first mixture via ion exchange and/or bipolar membrane electrodialysis to reduce a pH of the chelating composition to a pH of at least about 8, 8.5, 9, or 9.5. This process may be or include any one or more steps described in at least U.S. Pat. No. 8,551,312B2 which is expressly incorporated herein in its entirety in various non-limiting embodiments.

This method produces the chelating composition that includes the tetrasodium salt of glutamic acid N,N-diacetic acid in an amount of at least about 50 weight percent based on a total weight of the chelating composition as determined using an Fe-Total Sequestering Value; and wherein the chelating composition has a viscosity of less than about 1350 mPa·s measured at about 5° C., or less than about 350 mPa·s measured at about 20° C., each using a Brookfield DV II plus viscometer with spindle S18 and a temperature controlled bath. Any one or more of the aforementioned steps, compounds, mixtures, conditions, etc. may be alternatively as described above.

In one embodiment, the tetrasodium salt of glutamic acid N,N-diacetic acid present in an amount of from about 47 to about 60 weight percent based on a total weight of the chelating composition as determined using an Fe-Total Sequestering Value.

In another embodiment, the tetrasodium salt of glutamic acid N,N-diacetic acid present in an amount of greater than about 47 weight percent based on a total weight of the chelating composition as determined using an Fe-Total Sequestering Value.

In another embodiment, the first mixture has a pH of from about 11 to about 12 measured as an about 1 wt % solution.

In another embodiment, the composition is non-corrosive as determined by United Nations Standard Recommendations on the transport of the dangerous goods, Manual of tests and criteria; ST/SG/AC.10/11/Rev 4.

In another embodiment, the viscosity is less than about 1200 mPa·s at about 5° C. and the composition is classified as non-corrosive as determined by United Nations Standard Recommendations on the transport of the dangerous goods, Manual of tests and criteria; ST/SG/AC.10/11/Rev 4.

In another embodiment, the pH of the chelating composition is from about 9, 9.5 or 10 to about 11 measured as an about 1 wt % solution.

In other embodiments, other dosing profiles of HCN/formaldehyde are also possible, see for example U.S. Pat. No. 8,455,682, which is expressly incorporated herein by references in its entirety in various non-limiting embodiments. In one or more non-limiting embodiments, a low temperature in a nitrile reaction mix and a short residence time of the mononitrile are important aspects.

While at least one exemplary embodiment was presented in the foregoing detailed description, it should be appreciated that a vast number of variations exist. It should also be appreciated that the exemplary embodiment or exemplary embodiments are only examples, and are not intended to limit the scope, applicability, or configuration in any way. Rather, the foregoing detailed description will provide those skilled in the art with a convenient road map for implementing an exemplary embodiment. It being understood that various changes may be made in the function and arrangement of elements described in an exemplary embodiment without departing from the scope as set forth in the appended claims.

What is claimed is:

1. A method of forming a chelating composition, said method comprising the steps of:
providing a first mixture comprising water and the tetrasodium salt of glutamic acid N,N-diacetic acid and having a pH of greater than about 10; and
acidifying the first mixture via ion exchange and/or bipolar membrane electrodialysis to reduce a pH of the chelating composition to a pH of at least about 9;
wherein the chelating composition comprises the tetrasodium salt of glutamic acid N,N-diacetic acid in an amount of at least about 45 weight percent based on a total weight of the chelating composition as determined using an Fe-Total Sequestering Value; and
wherein the chelating composition has a viscosity of less than about 1350 mPa·s measured at about 5° C., or less than about 350 mPa·s measured at about 20° C., each using a Brookfield DV II plus viscometer with spindle S18 and a temperature controlled bath.

2. The method of claim 1 wherein the tetrasodium salt of glutamic acid N,N-diacetic acid present in an amount of from about 47 to about 60 weight percent based on a total weight of the chelating composition as determined using an Fe-Total Sequestering Value.

3. The method of claim 1 wherein the tetrasodium salt of glutamic acid N,N-diacetic acid present in an amount of greater than about 47 weight percent based on a total weight of the chelating composition as determined using an Fe-Total Sequestering Value.

4. The method of claim 1 wherein the first mixture has a pH of from about 11 to about 12 measured as an about 1 wt % solution.

5. The method of claim 2 wherein the first mixture has a pH of from about 11 to about 12 measured as an about 1 wt % solution.

6. The method of claim 3 wherein the first mixture has a pH of from about 11 to about 12 measured as an about 1 wt % solution.

7. The method of claim 1 that is non-corrosive as determined by United Nations Standard Recommendations on the transport of the dangerous goods, Manual of tests and criteria; ST/SG/AC.10/11/Rev 4.

8. The method of claim 2 that is non-corrosive as determined by United Nations Standard Recommendations on the transport of the dangerous goods, Manual of tests and criteria; ST/SG/AC.10/11/Rev 4.

9. The method of claim 3 that is non-corrosive as determined by United Nations Standard Recommendations on the transport of the dangerous goods, Manual of tests and criteria; ST/SG/AC.10/11/Rev 4.

10. The method of claim 4 that is non-corrosive as determined by United Nations Standard Recommendations on the transport of the dangerous goods, Manual of tests and criteria; ST/SG/AC.10/11/Rev 4.

11. The method of claim 1 wherein the viscosity is less than about 1200 mPa·s at about 5° C. and the composition is classified as non-corrosive as determined by United Nations Standard Recommendations on the transport of the dangerous goods, Manual of tests and criteria; ST/SG/AC.10/11/Rev 4.

12. The method of claim 2 wherein the viscosity is less than about 1200 mPa·s at about 5° C. and the composition is classified as non-corrosive as determined by United Nations Standard Recommendations on the transport of the dangerous goods, Manual of tests and criteria; ST/SG/AC.10/11/Rev 4.

13. The method of claim 3 wherein the viscosity is less than about 1200 mPa·s at about 5° C. and the composition is classified as non-corrosive as determined by United Nations Standard Recommendations on the transport of the dangerous goods, Manual of tests and criteria; ST/SG/AC.10/11/Rev 4.

14. The method of claim 4 wherein the viscosity is less than about 1200 mPa·s at about 5° C. and the composition is classified as non-corrosive as determined by United Nations Standard Recommendations on the transport of the dangerous goods, Manual of tests and criteria; ST/SG/AC.10/11/Rev 4.

15. The method of claim 1 wherein the pH of the chelating composition is from about 10 to about 11 measured as an about 1 wt % solution.

16. The method of claim 2 wherein the pH of the chelating composition is from about 10 to about 11 measured as an about 1 wt % solution.

17. The method of claim 3 wherein the pH of the chelating composition is from about 10 to about 11 measured as an about 1 wt % solution.

18. The method of claim 4 wherein the pH of the chelating composition is from about 10 to about 11 measured as an about 1 wt % solution.

19. The method of claim 5 wherein the pH of the chelating composition is from about 10 to about 11 measured as an about 1 wt % solution.

20. The method of claim 6 wherein the pH of the chelating composition is from about 10 to about 11 measured as an about 1 wt % solution.
